(19) United States
(12) Patent Application Publication
(10) Pub. No.: US 2024/0053361 A1
(43) Pub. Date: Feb. 15, 2024

(54) USE OF ALARMINS AS BIOMARKERS FOR ASSESSING ISCHEMIA-REPERFUSION INJURY SEVERITY AFTER SOLID ORGAN TRANSPLANTATION

(71) Applicants: INSTITUT NATIONAL DE LA SANTÉ ET DE LA RECHERCHE MÉDICALE, Paris (FR); CENTRE HOSPITALIER RÉGIONAL UNIVERSITAIRE DE TOURS, Tours (FR); CENTRE HOSPITALIER UNIVERSITAIRE DE POITIERS, Poitiers (FR); UNIVERSITÉ DE TOURS, Tours (FR); UNIVERSITE DE POITIERS, Poitiers (FR)

(72) Inventors: André HERBELIN, Poitiers (FR); Ephrem SALAME, Poitiers (FR); Jean-Marc GOMBERT, Poitiers (FR); Remy SINDAYIGAYA, Tours (FR); Aurelie ROBIN, Poitiers (FR); Louise BARBIER, Poitiers (FR)

(21) Appl. No.: 18/257,697

(22) PCT Filed: Dec. 14, 2021
(86) PCT No.: PCT/EP2021/085659

§ 371 Date: Jun. 15, 2023

(30) Foreign Application Priority Data
Dec. 16, 2020 (EP) 20306584.2

(51) Int. Cl. G01N 33/68

(52) U.S. Cl. CPC G01N 33/6875; G01N 2800/245; G01N 2800/56; G01N 2800/50; G01N 2333/54; G01N 2333/4703

(57) ABSTRACT

The inventors aim to assess IL-33 and HMGB1 contributions as alarmins to I/R injury following solid organ transplantation in particular liver transplantation. This study was conducted from a prospective biological collection and a clinical database of 40 liver transplant recipients in Tours hospital center. Serum IL-33 and HMGB1 levels were determined at graft reperfusion, at the end of the liver transplantation and at postoperative day 1 and 3. A post-reperfusion liver biopsy was systematic. Serum IL-33 increase is associated with: i) severe-moderate liver lesions; ii) an early allograft dysfunction; iii) a post-reperfusion syndrome occurrence; iv) a post liver transplantation acute kidney injury occurrence. Serum HMGB1 increase is associated with: i) an early allograft dysfunction; ii) a post-reperfusion syndrome occurrence. IL-33 and HMGB1 thus contribute as alarmins to I/R injury in human liver transplantation. Their serum levels are predictive of I/R injury severity and its clinical impact. Serum assay for IL-33 and HMGB1 upon graft reperfusion could be used as early biomarkers of early allograft function in solid organ transplantation, in particular in liver transplantation.

### FIELD OF THE INVENTION

The present invention is in the field of medicine, in particular solid organ transplantation.

### BACKGROUND OF THE INVENTION

Ischemia-reperfusion injury (IRI) upon transplantation contributes to graft damage¹ after a complex pathophysiology involving mitochondrial dysfunction, release of reactive oxygen species, cellular necrosis, apoptosis and tissue damage. It results in impaired organ function. Studies in IRI models have demonstrated that inflammatory responses mediated by the innate immune system cause damage. However, the mechanisms of early activation and recruitment of immune cells to the post-ischemic organ are still unclear, raising the question of possible involvement of proinflammatory damage-associated molecular patterns (DAMPs), which are host biomolecules that can initiate a noninfectious inflammatory response. DAMPs are normally intracellular, shielded from the immune system by plasma membranes, and their release, following tissue injury, signals cellular damage and activates the innate immune system. A subset of DAMPs, called alarmins, are tissue-derived nuclear proteins, which are constitutively expressed at high levels in epithelial barrier tissues and endothelial barriers. In particular, important pro-inflammatory functions have been ascribed to IL-33 and HMGB1, both as an alarmin. For instance, recent findings suggest that during kidney IRI, IL-33 acts as an alarmin promptly released into serum and urine after reperfusion. In this clinical situation, IL-33 levels and IRI duration are correlated, supporting a close connection between kidney cell injury and IL-33 release. However, the role of alarmins, an in particular the role of IL-33 and HMGB1, as biomarkers for assessing ischemia-reperfusion injury severity after solid organ transplantation has never been investigated.

### SUMMARY OF THE INVENTION

The present invention is defined by the claims. In particular the present invention relates to use of alarmins as biomarkers for assessing ischemia-reperfusion injury severity after solid organ transplantation.

### DETAILED DESCRIPTION OF THE INVENTION

The first object of the present invention relates to a method for assessing ischemia-reperfusion injury severity after solid organ transplantation comprising determining the level of at least one alarmin in a sample obtained from the transplant patient wherein said level correlates with the ischemia-reperfusion injury severity.

In some embodiment, the level positively correlates with the ischemia-reperfusion injury severity.

In some embodiments, the patient can be human or any other animal (e.g., mammals). Typically said patient is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the patient is a human. In some embodiments, the patient is a human infant. In some embodiments, the patient is a human child. In some embodiments, the patient is a human adult. In some embodiments, the patient is an elderly human. In some embodiments, the patient is a premature human infant.

As used herein, the term “solid organ transplantation” and variations thereof refers to the insertion of a solid organ (also called graft) into a recipient, whether the transplantation is syngeneic (where the donor and recipient are genetically identical), or allogeneic (where the donor and recipient are of different genetic origins but of the same species).

As used herein, the term “solid organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

In some embodiments, the transplanted organ comes from a deceased donor, either after circulatory death or brain death. In some embodiments, the transplanted organ is a living organ donation.

The method of the present invention is particularly suitable in the context of liver transplantation.

As used herein, the term “liver transplantation” has the common meaning in the art and includes partial and whole liver transplantation in which a liver of a donor is partially or wholly resected and partially or wholly transplanted into a recipient. Partial liver transplantation is classified by operation mode into orthotopic partial liver transplantation, heterotopic partial liver transplantation, and the like, and the present invention can be applied to any of them. In partial liver transplantation, a liver transplant or a partial liver transplant from a donor corresponding to about 30-50% of the normal liver volume of a recipient is typically transplanted as a graft into the recipient whose liver has been wholly resected.

As used herein, the term “ischemia” as used herein refers to a restriction in blood supply with resultant damage or dysfunction of the organ. Rather than hypoxia (a more general term denoting a shortage of oxygen, usually a result of lack of oxygen in the air being breathed), ischemia is an absolute or relative shortage of the blood supply to an organ, i.e. a shortage of oxygen, glucose and other blood-borne components. A relative shortage means the mismatch of blood supply (oxygen/fuel delivery) and blood request for adequate metabolism of tissue.

In some embodiments, the transplanted organ is the subject of a warm ischemia and/or cold ischemia.

As used herein, the term “warm ischemia” has its general meaning in the art and is used to describe ischemia of cells and tissues under normothermic conditions.

As used herein, the term “cold ischemia” has its general meaning in the art and refers to the organ chilling during decreased blood perfusion or in the absence of blood supply.

As used herein, the term “reperfusion” has its general meaning in the art and refers to the restoration of blood flow to a tissue following ischemia.

Accordingly, as used herein, the term “ischemia reperfusion” or “PR” is thus intended to encompass an event wherein an episode of ischemia is followed by an episode of reperfusion.

As used herein, the term “ischemia reperfusion injury” or “I/R injury” refers to the tissue damage caused by an ischemia reperfusion event. The absence of oxygen and nutrients from blood during the ischemic period creates a condition in which the restoration of circulation results in inflammation and oxidative damage through the induction of oxidative stress rather than (or along with) restoration of normal function.

As used herein, the term “ischemia reperfusion injury severity” or “I/R injury severity” refers to a measure of the degree of injury.

The method of the present invention is particularly suitable for determining whether the transplant patient is at risk of an early graft rejection.

Accordingly, a further object of the present invention relates to a method of determining whether a transplant patient is a risk of early allograft dysfunction after solid organ transplantation comprising determining the level of at least one alarmin in a sample obtained from the transplant patient wherein said level indicates the risk of early allograft dysfunction (EAD) and/or primary graft non function (PNF).

As used herein, the term “early allograft dysfunction” or “EAD” has its general meaning in the art and refers to the medical condition after organ transplantation characterized by the absence or incompleteness of the respective organ functions compared to a transplant not suffering from this condition. The term thus defines grafts with marginal function early after transplantation. It has been validated in the EAD is associated with increased recipient susceptibility to sepsis, longer intensive care unit (ICU) and hospital stays, graft loss, and greater morbidity and mortality.

As used herein, the term “primary graft non function” (PNF) is defined as an early and irreversible graft failure with no technical complications or immunological disorders. It is the most severe complication after liver transplantation and conducts ineluctably to death in the absence of early retransplantation.

As used herein, the term “extended criteria donors” (ECD) is a subjective evaluation of the surgeon based on an association of multiple factors as donor age, altered liver function tests, donors after circulatory death, cold ischemia time, which eludes to severe reperfusion injury and early poor liver function.

As used herein, the term “risk” in the context of the present invention, relates to the probability that an event will occur over a specific time period and can mean a subject's “absolute risk” or “relative risk”. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk.

In some embodiments, the sample is any sample liable to contain an alarmin. Non limiting examples include blood and urine samples. In some embodiments, the sample is a blood sample.

As used herein the term “blood sample” means any blood sample derived from the subject. Collections of blood samples can be performed by methods well known to those skilled in the art. In some embodiments, the blood sample is a serum sample or a plasma sample.

As used herein, the term “alarmin” refers to any molecule released from a damaged or diseased cell that stimulates an immune response. Non-limiting examples of alarmins are heat-shock proteins, interleukin-la, HMGB1, IL-33 and nucleosomes.

In some embodiments, the level of IL-33 and/or HMGB1 is determined in the sample obtained from the transplant patient.

In some embodiments, the level of IL-33 is determined in the sample obtained from the transplant patient.

As used herein, the term “IL-33” has its general meaning in the art and refers to the human IL-33 protein having the amino acid sequence as set forth in NCBI accession Nos. NP 254274.1 (human isoform 1), NP 001186569.1 (human isoform 2), or NP 001186570.1 (human isoform 3).

In some embodiment, the level of HMGB1 is determined in the sample obtained from the transplant patient.

As used herein, the term “HMGB1” has its general mean in the art and refers to a family member of the S100 calcium binding proteins and includes all know HMGB1 molecules including human, naturally occurring variants and those deposited in Genbank, for example, with accession number NM 002128.4, each of which is herein incorporated by reference.

In some embodiments, both levels of IL-33 and HMGB1 are determined in the sample obtained from the transplant patient.

Typically, the level of the alarmin is determined by an immunoassay. Such assays include, for example, competition assays, direct reaction assays sandwich-type assays and immunoassays (e.g. ELISA). The assays may be quantitative or qualitative. There are a number of different conventional assays for detecting formation of an immunocomplex. For example, the detecting step can comprise performing an ELISA assay, performing a lateral flow immunoassay, performing an agglutination assay, analyzing the sample in an analytical rotor, or analyzing the sample with an electrochemical, optical, or opto-electronic sensor. These different assays are well-known to those skilled in the art. In some embodiments, the devices are useful for performing an immunoassay according to the present invention. For example, in some embodiments, the device is a lateral flow immunoassay device. In some embodiments, the device is an analytical rotor. In some embodiments, the device is a dot blot. In some embodiments, the device is a tube or a well, e.g., in a plate suitable for an ELISA assay. In some embodiments, the device is an electrochemical sensor, an optical sensor, or an opto-electronic sensor. The presence and amount of the immunocomplex may be detected by methods known in the art, including label-based and label-free detection. For example, label-based detection methods include addition of a secondary antibody that is coupled to an indicator reagent comprising a signal generating compound. The secondary antibody may be an anti-human IgG antibody. Indicator reagents include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. Methods of label-free detection include surface plasmon resonance, carbon nanotubes and nanowires, and interferometry. Label-based and label-free detection methods are known in the art and disclosed, for example, by Hall et al. (2007) and by Ray et al. (2010) Proteomics 10:731-748. Detection may be accomplished by scanning methods known in the art and appropriate for the label used, and associated analytical software. In some embodiments, fluorescence labeling and detection methods are used to detect the immunocomplexes. A particularly useful assay format is a lateral flow immunoassay format. Antibodies to human or animal (e.g., dog, mouse, deer, etc.) immunoglobulins, or staph A or G protein antibodies, can be labeled with a signal generator or reporter (e.g., colloidal gold) that is dried and placed on a glass fiber pad (sample application pad or conjugate pad). Another assay is an enzyme linked immunosorbent assay, i.e., an ELISA. Typically, in an ELISA, the alarmins are adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of nonfat dry milk which also contains a preservative, salts, and an antifoaming agent). The well is then incubated with the sample. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody (e.g., for human subjects, an anti-human immunoglobulin (αHuIg) from another animal, such as dog, mouse, cow, etc. that is conjugated to an enzyme or other label by standard procedures and is dissolved in blocking buffer. The label can be chosen from a variety of enzymes, including horseradish peroxidase (HRP), beta-galactosidase, alkaline phosphatase, glucose oxidase, etc. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length).

Typically, high levels of alarmin (e.g. IL-33 and/or HMGB1) indicate that the I/R injury is severe and/or indicate a high risk of EAD and/or PNF and conversely low levels of alarmin indicate that the I/R injury is mild and/or indicate a low risk of EAD and/or PNF.

As used herein, the term “high” refers to a measure that is greater than normal, greater than a standard such as a predetermined reference value or a subgroup measure or that is relatively greater than another subgroup measure. For example, high levels of alarmin refers to a level of alarmin that is greater than a normal alarmin level. A normal alarmin level may be determined according to any method available to one skilled in the art. High level of alarmin may also refer to a level that is equal to or greater than a predetermined reference value, such as a predetermined cutoff High level of alarmin may also refer to a level of alarmin wherein a high alarmin subgroup has relatively greater levels of alarmin than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. In some cases, a “high” level may comprise a range of level that is very high and a range of level that is “moderately high” where moderately high is a level that is greater than normal, but less than “very high”.

As used herein, the term “low” refers to a level that is less than normal, less than a standard such as a predetermined reference value or a subgroup measure that is relatively less than another subgroup level. For example, low level of alarmin means a level of alarmin that is less than a normal level of in a particular set of samples of patients. A normal level of alarmin measure may be determined according to any method available to one skilled in the art. Low level of alarmin may also mean a level that is less than a predetermined reference value, such as a predetermined cutoff. Low level of alarmin may also mean a level wherein a low level alarmin subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high (i.e., greater than the median).

In some embodiments, the method of the present invention further comprises comparing the level of alarmin with a predetermined reference value wherein detecting a difference between the level of alarmin and the predetermined reference value indicates the severity of the FR injury and/or whether the subject is or is not at risk of having EAD and/or PNF.

Thus, in other words, the present invention relates to a method for assessing ischemia-reperfusion injury severity after solid organ transplantation comprising: i) determining the level of at least one alarmin in a sample obtained from the transplant patient, ii) comparing the level of at least one alarmin determined in step i) with a predetermined reference value; and iii) concluding that the ischemia-reperfusion injury is severe when the level determined at step i) is higher than the predetermined reference value.

The present invention relates to a method of determining whether a transplant patient is a risk of early allograft dysfunction after solid organ transplantation comprising: i) determining the level of at least one alarmin in a sample obtained from the transplant patient, ii) comparing the level of a least one alarmin determined in step i) with a predetermined reference value; and iii) concluding that the subject is at risk of having early allograft dysfunction (EAD) and/or primary graft non function (PNF) when the level determined at step i) is higher than the predetermined reference value.

In some embodiments, the predetermined reference value is a relative to a number or value derived from population studies, including without limitation, subjects of the same or similar age range, subjects in the same or similar ethnic group, and subjects having the same severity of I/R injury. Such predetermined reference values can be derived from statistical analyses and/or risk prediction data of populations obtained from mathematical algorithms and computed indices. In some embodiments, retrospective measurement of the level of the alarmin in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the alarmin in a group of reference, one can use algorithmic analysis for the statistic treatment of the measured levels of the alarmin in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method of the present invention further comprises a step consisting of calculating a score, representing an estimation of FR injury severity and/or risk of EAD and/or PNF. Typically, the score is based on the level of the level of alarmin determined in the sample and may typically include another factor. Typically, other risk factors may include additional features such as age, gender, obesity, diabetes mellitus, smoking, body mass index . . . . In some embodiments, the other risk factor is the MEAF score (Model for Early Allograft Function Scoring). Based the above input features obtained from the subject, an operator can calculate a numerical function of the above list of inputs by applying an algorithm. For instance, this numerical function may return a number, i.e. score (R), for instance between zero and one, where zero is the lowest possible risk indication and one is the highest. This numerical output may also be compared to a threshold (T) value between zero and one. If the risk score exceeds the threshold T, it is meant than the patient has a severe I/R injury and/or a high risk of EAD and/or PNF and if the risk score is under the threshold T, it is meant than the patient has a mild I/R injury and/or a low risk of EAD and/or PNF.

In some embodiments, the method of the invention thus comprises the use of an algorithm. In some embodiments, the algorithm is a classification algorithm typically selected from Multivariate Regression Analysis, Linear Discriminant Analysis (LDA), Topological Data Analysis (TDA), Neural Networks, Support Vector Machine (SVM) algorithm and Random Forests algorithm (RF). As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the subject trees. The subject trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention thus comprises a) determining the level of alarmin in the sample obtained from the subject; b) implementing an algorithm on data comprising the level of alarmin so as to obtain an algorithm output; c) determining the I/R injury severity and/or the risk of EAD and/or PNF.

In some embodiments, the methods of the present invention is performed in vitro or ex vivo

The invention will be further illustrated by the following figures and examples. However, these examples and figures should not be interpreted in any way as limiting the scope of the present invention.

### FIGURES

FIG. 1: IL-33 (A) and HMGB1 (B) are released immediately after liver allograft reperfusion and act as alarmins in the context of hepatic ischemia reperfusion injury. ROC curve of IL-33 (C) and HMGB1 (D) as early test of early allograft function (MEAF). AUROC=“area under the ROC curve. Data are expressed as mean±SEM. ***p<0.0001, ns: not significant by Friedman or Mann-Whitney test, as appropriate. C: healthy controls (n=15), TO: patients prior to liver transplantation (n=40), T1: patients at portal vein unclamping (n=40), T2: patients at the end of liver transplantation (n=40).

FIG. 2: IL-33 and HMGB1 serum levels at portal vein unclamping are associated with I/R injury severity (A) measured by a predefined histopathological scoring and its systemic impact (B-G): MEAF (Model for Early Allograft Function) score defined by Paréja et al. (Liver Transplantation, 2015) (B-C); post reperfusion syndrome as I/R vascular's impact criteria defined by Aggarwal et al. (journal of critical care, 1993) (D-E); I/R renal's impact defined by the KDIGO grading system (Jochmans et al., liver transplantation, 2017) (F-G). Data are expressed as mean±SEM. Mann-Whitney test.

### EXAMPLE

Background: IL-33's contribution as an alarmin was recently reported in renal I/R (Thierry et al., Plos One, 2014) and in a murine model of warm hepatic ischemia (Barbier et al., European society of organ transplantation, 2019). We aim to assess IL-33′ and HMGB1′ contributions as alarmins to I/R injury or EAD/PNF following liver transplantation.

Methods: This study was conducted from a prospective biological collection and a clinical database of 40 liver transplant recipients in Tours hospital center. Serum IL-33 and HMGB1 levels were determined at graft reperfusion, at the end of the liver transplantation and at postoperative day 1 and 3. A post-reperfusion liver biopsy was systematic.

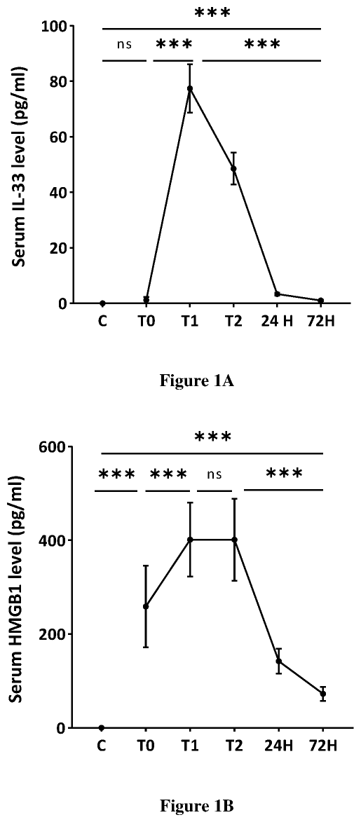

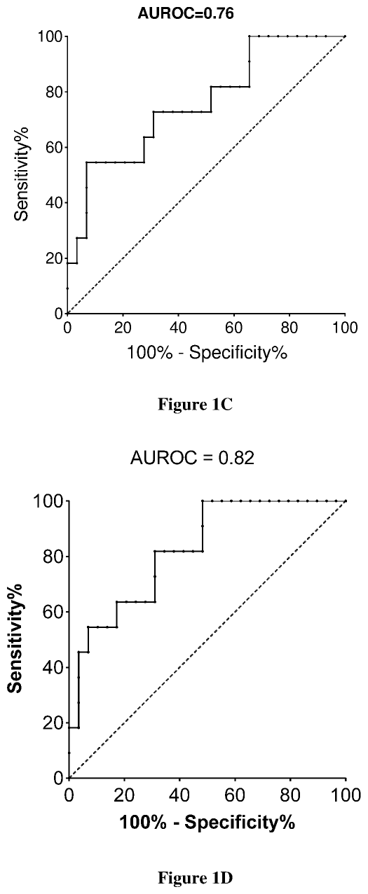

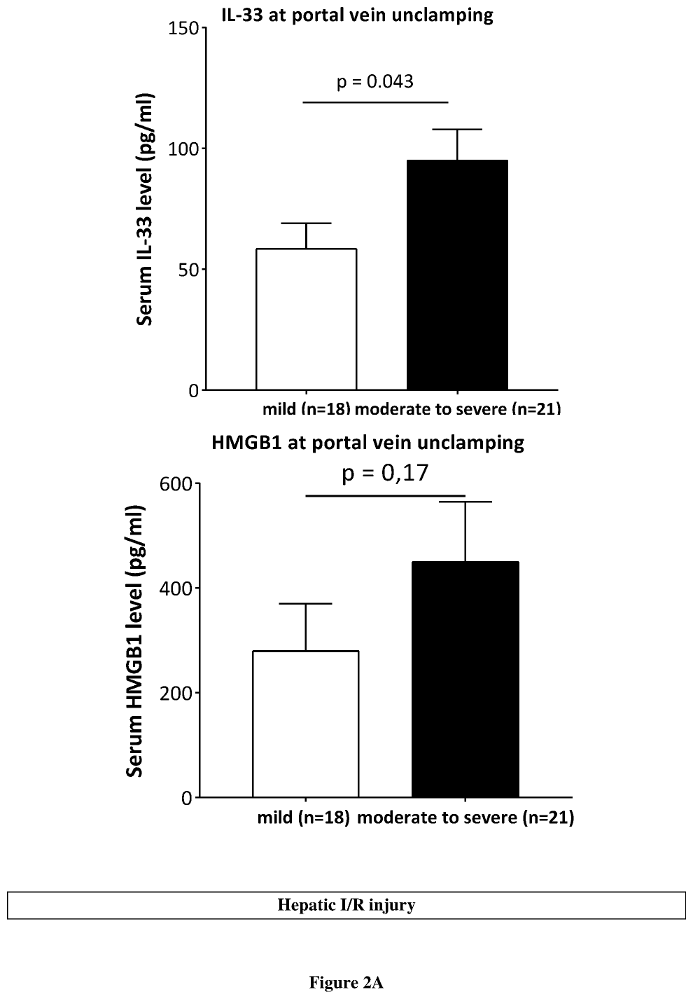

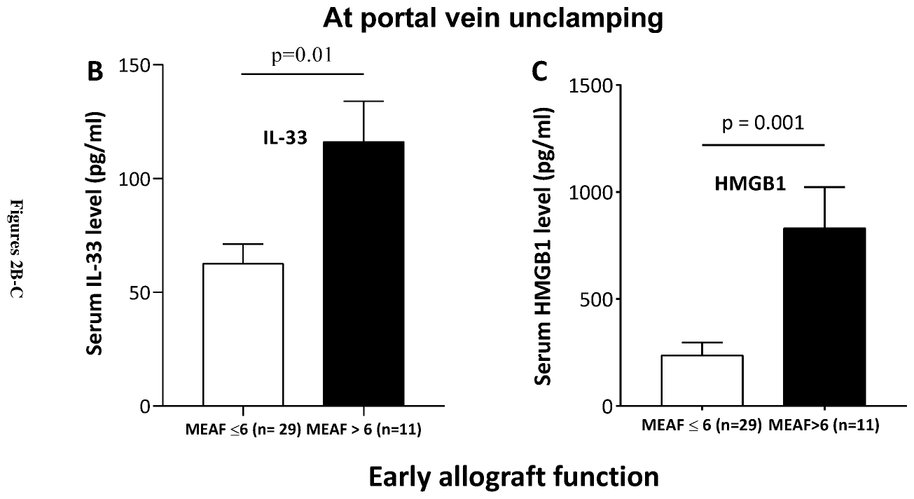

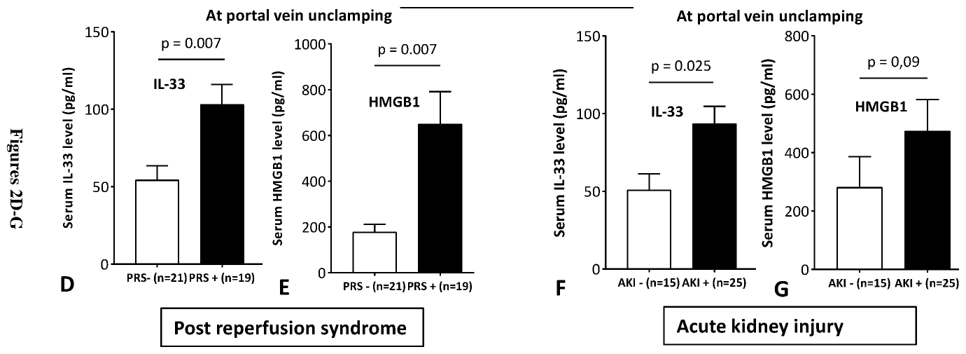

Results: Serum IL-33 level peaks at 77 pg/mL (±9) immediately upon graft reperfusion. Serum HMGB1 level increase upon graft reperfusion to reach its peak of 243 pg/mL (±19) at the end of LT. Serum IL-33 increase is associated with: i) severe-moderate liver lesions (AUC=140 (81-212) vs 87 (66-135) in the presence of mild liver lesions; p=0.043); ii) an early allograft dysfunction (AUC=195.5 (88.26-291.3) in the presence of a MEAF score >6 vs 88.30 (57.6-150.3) in the presence of a MEAF score ≤6; p=0.01); iii) a post-reperfusion syndrome occurrence (AUC=85 (29-128) vs 160 (88-268) in the absence and in the presence of such syndrome, respectively; p=0.007); iv) a post liver transplantation acute kidney injury occurrence (AUC=87 (28-107) vs 134 (86-207) in the absence and in the presence of an acute kidney injury, respectively; p=0.025). Serum HMGB1 increase is associated with: i) an early allograft dysfunction (AUC=1588 (1243-3080) in the presence of a MEAF score >6 vs 531 (272-943) in the presence of a MEAF score ≤6; p=0.0002); ii) a post-reperfusion syndrome occurrence (AUC=504 (9.42-1430) vs 1292 (138-5325) in the absence and in the presence of such syndrome, respectively; p=0.005). A ROC curve was used to determine the performance of IL-33 and HMGB1 as diagnostic tests for recovery of function. The area under the ROC curve was 0.76 (p=0.01) and 0.82 (p=0.0017) for IL-33 and HMGB1, respectively. An optimal threshold value (73.0 pg/mL and 278.1 pg/mL for IL-33 and HMGB1, respectively) predictive of graft recovery was determined by calculation of the Youden index. The results are depicted in FIGS. 1 and 2.

Conclusion: IL-33 or HMGB1 contribute as alarmins to I/R injury in human liver transplantation. Their serum levels are predictive of I/R injury severity and its clinical impact. Serum IL-33 and HMGB1 assays upon graft reperfusion could be used as early biomarkers of early allograft function or primary graft non function.

### REFERENCES

Throughout this application, various references describe the state of the art to which this invention pertains. The disclosures of these references are hereby incorporated by reference into the present disclosure.

1. A method for assessing ischemia-reperfusion (I/R) injury severity and/or a risk of early allograft dysfunction (EAD) after solid organ transplantation and treating the I/R injury and/or the EAD in a transplant patient in need thereof, comprising determining the level of at least one alarmin in a sample obtained from the transplant patient and performing a retransplantation in a subject identified as having a high level of the at least one alarmin, compared to a predetermined reference value.

2. (canceled)

3. The method of claim 1 wherein the solid organ is selected from the group consisting of heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, and uterus.

4. The method of claim 1 wherein the solid organ transplantation is liver transplantation.

5. The method of claim 1 wherein the sample is a blood sample.

6. The method of claim 1 wherein the level of IL-33 is determined in the sample obtained from the transplant patient.

7. The method of claim 1 wherein the level of HMGB1 is determined in the sample obtained from the transplant patient.

8. The method of claim 1 wherein levels of both IL-33 and HMGB1 are determined in the sample obtained from the transplant patient.

9. The method of claim 1 wherein the level of the at least one alarmin is determined by an immunoassay.

10. The method of claim 1 wherein high levels of the at least one alarmin indicate that the I/R injury is severe and/or indicate a high risk of EAD and/or PNF and conversely low levels of alarmin indicate that the I/R injury is mild and/or indicate a low risk of EAD and/or PNF.

11. The method of claim 1 that comprises comparing the level of the at least one alarmin with a predetermined reference value wherein detecting a difference between the level of the at least one alarmin and the predetermined reference value indicates the severity of the I/R injury and/or whether the subject is or is not at risk of having EAD and/or PNF.

12. The method of claim 1 wherein the method further comprises calculating a score representing an estimation of I/R injury severity and/or risk of EAD and/or PNF.

13. The method of claim 1 that comprises a) determining the level of the at least one alarmin in the sample obtained from the subject; b) implementing an algorithm on data comprising the level of the at least one alarmin so as to obtain an algorithm output; and c) determining the I/R injury severity and/or the risk of EAD and/or PNF based on the algorithm output.

14. A method, comprising
transplanting a solid organ into a transplant patient in need thereof, and
determining a level of at least one alarmin in a sample obtained from the transplant patient.

15. The method of claim 14, further comprising identifying the transplant patient as having a level of at least one alarmin that is higher than a predetermined reference value and treating the transplant patient.

16. A method, comprising
transplanting a solid organ into a transplant patient in need thereof, and
determining that a level of at least one alarmin in a sample obtained from the transplant patient is higher than a predetermined reference value.

17. The method of claim 16, further comprising treating the transplant patient by retransplantation.

18. The method of claim 9, wherein the immunoassay is an ELISA assay.
